(19) United States
(12) Patent Application Publication
(10) Pub. No.: US 2013/0052715 A1
(43) Pub. Date: Feb. 28, 2013

(54) METHOD FOR PRODUCING RECOMBINANT THROMBIN

(71) Applicants: Andreas Anton, Halle (DE); Arndt Dietrich, Halle (DE); Jochen Koetter, Halle (DE); Joerg Schaeffner, Halle (DE)

(72) Inventors: Andreas Anton, Halle (DE); Arndt Dietrich, Halle (DE); Jochen Koetter, Halle (DE); Joerg Schaeffner, Halle (DE)

(73) Assignee: Scil Proteins GmbH, Halle/Saale (DE)

(21) Appl. No.: 13/641,844

(22) PCT Filed: Apr. 20, 2011
(86) PCT No.: PCT/EP2011/056359

§ 371 Date: Oct. 17, 2012

(30) Foreign Application Priority Data
Apr. 22, 2010 (EP) 10160740.6

(51) Int. Cl. C12N 9/74

(52) U.S. Cl. 435/214

(57) ABSTRACT

The invention relates to a method for producing folded prethrombin, wherein inclusion bodies, which contain unfolded prethrombin or a derivative thereof, are solubilized in a solubilization buffer containing at least one chaotropic compound and at least one organic disulfide compound. The invention further relates to methods for producing thrombin and a-thrombin and derivatives thereof. The invention also relates to solutions that contain folded proteins, which can be produced by the methods according to the invention.

The invention provides methods for producing folded prethrombin and thrombin from inclusion bodies. The invention also provides solutions comprising folded proteins which can be produced by the method according to the invention.

### PRIOR ART

The central enzymes of the blood coagulation cascade are serine proteases, which are synthesized in the liver as somewhat larger inactive precursors, known as zymogens. They include prothrombin, the inactive precursor of thrombin, which is also called factor II. Thrombin plays a central role in the blood coagulation cascade and fibrinolysis. The central function in blood clotting makes thrombin of interest for use in human medicine. It has been used as a medicament for a long time, for example in order to close wounds as rapidly as possible (Zymogenetics, Inc. “Annual Report on Form 10-K For the Year Ended Dec. 31, 2003” 2003; Nakajima et al. Ann. Thorac. Surg. 2005 79: 1793-1794).

In addition, thrombin is also involved in many other processes. Thus it initiates, for example, the discontinuation of prothrombin activation and the start of fibrinolysis (Esmon & Jackson Journal of Biological Chemistry. 1974 249(24): 7791-7797). Various studies have furthermore shown that thrombin is involved in embryogenesis, carcinogenesis, regulation of vascular pressure, Alzheimer's disease and wound healing processes (Grand et al. Biochemical Journal.” 1996 313(2): 353-368; Tsopanoglou & Maragoudakis Journal of Biological Chemistry. 1999 274(34): 23969-23976). Because of its high polyfunctionality, thrombin is a very interesting active compound for medical and biotechnological uses. It is employed for cleavage of fusion proteins and therefore for removal of purification and assay tags (Jenny et al., Protein Expression and Purification, 2003 31(1): 1-11.).

On the basis of its homology with other proteases, such as chymotrypsin, trypsin and elastase, thrombin is assigned to the class of serine proteases. The serine proteases have in common the same catalysis mechanism. The active centre of serine proteases is characterised by the catalytic triads of serine-histidine-aspartate typical of them. Thrombin consists of two chains, the A and the B chain, which are bonded to one another via a disulphide bridge. The shorter A chain of thrombin shows no common features with the pancreatic enzymes trypsin, chymotrypsin and elastase. On the other hand, the B chain of these serine proteases, which has three further intramolecular disulphide bridges, scarcely differs (Bode et al. Protein Science. 1992 1(4): 426-471. Stubbs & Bode Thrombosis Research. 1993 69(1): 1-58 Stubbs & Bode Trends Biochem Sci. 1995 January; 20(1):23-8. Review. Erratum in: Trends Biochem Sci 1995 March; 20(3):131). Like all serine proteases, thrombin essentially also consists of two six-stranded 8-folded leaves, each of which is rolled together into a barrel-like structure. The V-shaped A chain runs in a groove recessed through the larger B chain on the side opposite the active centre.

The associated zymogen prothrombin (72 kDa) consists of a Gla domain, 2 curled domains and an A and a B chain (Hiller 2003, Dissertation to obtain a sciences doctorate degree of the University of Bielefeld, Butenas & Mann Biochemistry, Moscow, Russian Federation, 2002, 67(1): 3-12). The two A and B chains are bonded in the active protein via a disulphide bridge. Prothrombin is cleaved between Arg₂₇₁-Thr₂₇₂ and between Arg₃₂₀-Ile₃₂₁ by the prothrombinase complex. The N terminus is thereby removed and the A and B chain are separated from one another. Depending on the sequence of this activation, various intermediate products, meizothrombin and prethrombin-2, are formed (Rosing et al. Journal of Biological Chemistry, 1986 261: 4224-4228.).

The use of recombinant thrombin (rh-thrombin) as an active compound is an appropriate alternative to the products currently on the market which are obtained from bovine and human blood plasma. On the one hand, with the use of rh-thrombin the risk of transmission of infectious diseases, such as, for example, HIV and hepatitis B, can be reduced to a minimum. On the other hand, an immune response is scarcely to be expected in humans, since this is an active compound which has very similar features to endogenous α-thrombin. Furthermore, a considerable reduction in costs is to be expected with recombinant production compared with conventional processes, since as a rule very high product yields can be achieved in high cell density fermentation processes.

Various methods for recombinant production of α-thrombin in eukaryotic and prokaryotic cells are described in the literature. In most of the methods α-thrombin is obtained from the inactive precursor molecule prethrombin-2, which is activated after a first chromatographic purification step by the metalloprotease ecarin (Russo et al., 1997, Protein Expr. Purif., 10(2):214-25; Yonemura et al., 2004, J. Biochem. (Tokyo), 135(5):577-82; Soejima et al., Journal of Biochemistry, 2001, 130(2): 269-277). For example, ZymoGenetics Inc. has developed a production process for rh-thrombin in eukaryotic CHO cells, wherein prethrombin-1 is used as a starting precursor molecule.

The production of recombinant proteins in mammalian cells as a rule is costly and time-consuming and delivers moderate product yields. In the known eukaryotic fermentation processes, product yields of from 25 to 140 mg/l of fermentation medium are achieved. In this case, the target protein prethrombin-2 is secreted into the medium (Russo et al. 1997, Yonemura et al. 2004).

To improve these critical parameters, production of prokaryotes is an alternative.

Soejima et al., 2001 disclose a method for expression of prethrombin-2 in Escherichia coli (E. coli) with subsequent steps for solubilization and folding. Only low product yields of folded native prethrombin-2 are achieved. Compared with the yields which are achieved in eukaryotic expression systems, the process described by Soejima et al. 2001 proves to be disadvantageous. Detergents, such as Triton X-100 or Brij-58, are employed in the method described by Soejima et al., 2001.

JP 2002 306163 discloses a method for producing folded thrombin. The method includes the expression in E. coli, the obtaining of the inclusion bodies and the solubilization thereof in solubilization buffer. Detergents, such as e.g. Tween, Brij or Triton, are furthermore used.

### OBJECT OF THE INVENTION

The invention is based on the object of providing a method for producing folded prethrombin and thrombin which overcomes the disadvantages described above.

The invention is based in particular on the object of developing a method for producing thrombin and prethrombin which is easy to carry out and achieves a high yield.

In this context, the method should render it possible to produce folded prethrombin and thrombin in high yields and in a high purity. The method should preferably use a prokaryotic expression system.

### SUBJECT MATTER OF THE INVENTION

The object is achieved, surprisingly, by methods according to the claims.

The invention provides a method for producing folded prethrombin or a derivative thereof, wherein inclusion bodies which comprise non-folded prethrombin or a derivative thereof are solubilized in a solubilization buffer which comprises at least one chaotropic compound and at least one organic disulphide compound.

The method according to the invention serves for the production and purification of human prethrombin. Prethrombin-2 is preferably employed. The protein has 309 amino acids and a molecular weight of 35,485.5 Da. The amino acid sequence is shown in FIG. 3. Human prethrombin can be cleaved to give α-thrombin, which has 295 amino acids and a molecular weight of 33,810.7 Da. The sequence and the A and B chain are shown in FIG. 4.

According to the invention, derivatives of prethrombin can also be employed. These are derivatives of which the processed form has a thrombin-like activity, in particular protease activity. The derivatives of prethrombin are corresponding precursor polypeptides, from which the active thrombin derivatives can be obtained by proteolytic cleavage. The derivatives are, in particular, those which can be obtained by amino acid mutation, deletion or insertion or by chemical modification of the polypeptide. Preferably, the derivatives have a sequence identity to the wild-type protein of more than 80%, more than 90% or more than 95%. The derivatives have at least one cysteine per polypeptide chain, but preferably all native cysteines. The person skilled in the art can see that the method according to the invention can also be carried out generally with derivatives of prethrombin. After the folding and splitting off of prepeptides, the corresponding derivatives of thrombin or α-thrombin are obtained. Statements which are made in the following with respect to the methods and in which reference is made to prethrombin, thrombin and α-thrombin therefore apply equally to the derivatives of these polypeptides. In the following general explanations, the term “prethrombin” can thus be equated with “prethrombin or derivatives of prethrombin”. This applies equally to thrombin and α-thrombin.

In the method according to the invention, inclusion bodies are solubilized. Inclusion bodies (IB) are accumulations of usually defective or incompletely folded proteins. They form inside cells, for example bacteria cells, such as E. coli, in the event of excessive expression of recombinant proteins. The inclusion bodies employed according to the invention preferably comprise the folded prethrombin in a high purity. This means that they contain at least 60, at least 70, at least 80 or at least 90 wt. % of prethrombin (based on the total amount of protein).

The solubilization buffer comprises a disulphide compound. The disulphide compound is capable of forming mixed disulphides with thiol groups (—SH) of cysteines of the polypeptides in the inclusion bodies. The disulphide is added to the solution. The disulphide does not designate proteins which the inclusion bodies comprise and which possibly comprise disulphide bridges. Preferably, the disulphide is not a true peptide. Preferably, the disulphide is a low molecular weight compound. The molecular weight is, for example, lower than 2,000 g/mol or than 1,000 g/mol. The disulphide is employed, for example, in a concentration of from 5 mM to 1M, in particular 10 mM to 0.5 M.

In a preferred embodiment of the invention, the disulphide compound is glutathione disulphide. Glutathione (GSH), also y-L-glutamyl-L-cysteinylglycine, is a pseudo-tripeptide which is formed from the three amino acids glutamic acid, cysteine and glycine. GSH is present in the cytoplasm of both prokaryotes and eukaryotes and is involved in the formation of disulphide bridges. It is in equilibrium with the dimer GSSG, which contains a disulphide bridge. Glutathione reacts with cysteines R-SH and R′-SH from two polypeptides or from a single polypeptide in a disulphide exchange reaction:

R-SH+GSSG→R-S-S-G+GSH.

RSSG is called a mixed disulphide. It is reacted with a further cysteine of a polypeptide, so that as a result a disulphide bridge is obtained between two cysteines:

R-S-S-G+HS-R′→R-S-S-R′+GSH.

Glutathione is kept enzymatically in the reduced form (GSH) in the cytosol. “Reducing conditions” in the cytosol are therefore referred to. Conditions are established in the solubilization buffer so that the disulphide compound it comprises catalyses the formation of disulphide bridges in accordance with the reactions described above. The GSSG is employed, for example, in a concentration of from 10 mM to 0.5 M.

The solubilization buffer comprises at least one chaotropic substance. Chemical substances which dissolve ordered hydrogen bridge bonds in water are called chaotropic. Since the hydrogen bridge bonds are broken open, the chaotropic substances interfere with the water structure and ensure disorder (increase in entropy). The reason for this is that the formation of the H₂O cage structures necessary for the solvation is prevented. In the case of amino acids, they reduce the hydrophobic effects and have a denaturing action on proteins, since a driving force of protein folding is the assembling together of hydrophobic amino acids in water. Generally, any substance which exerts the hydrophobic effect in the solubilization buffer and therefore has a denaturing action on the proteins can be employed as a chaotropic substance. Chaotropic substances are in general salts or low molecular weight compounds, such as urea. Chaotropic substances are clearly distinguished from detergents, since they contain no hydrophobic radical, such as an alkyl radical, in the molecule. Generally, the chaotropic action is accompanied by an improvement in the solubility of the protein, in this case the prethrombin.

In a preferred embodiment of the invention, the chaotropic compound is chosen from guanidinium salts, in particular guanidinium hydrochloride and guanidinium thiocyanate, iodides, barium salts, thiocyanates, urea and perchlorates.

The chaotropic compounds are employed in conventional amounts. For example, 4-8 M guanidinium hydrochloride or 4-9 M urea can be employed.

In a preferred embodiment of the invention, the solubilization buffer is a Tris buffer. In a preferred embodiment of the invention, the reagent which can form mixed disulphides with the —SH groups of the protein in the inclusion bodies is GSSG and the chaotropic substance is guanidinium hydrochloride.

The solubilization buffer can comprise further conventional additives, for example EDTA or salts. The pH of the solubilization buffer is, for example, between 6 and 11, preferably between 7 and 10. The solubilization is preferably assisted mechanically, for example with conventional homogenization apparatuses or by means of ultrasound. After the solubilization, solids which remain are preferably separated off. The supernatant comprises the solubilized prethrombin.

In a preferred embodiment of the invention, the inclusion bodies have been obtained by recombinant expression of prethrombin in prokaryotic cells. In a preferred embodiment of the invention, the prokaryotic host cell is a bacterium, preferably E. coli. In a preferred embodiment of the invention, the bacterium is E. coli JM108 (DSMZ 5585; 3.3.2006), preferably transformed with a plasmid originating from pSCIL008.

Preferably, after the solubilization excess disulphide compound is removed. The removal of the reagent is preferably carried out using a method for buffer exchange, that is to say via dialysis, chromatography or tangential flow filtration.

In a preferred embodiment of the invention, the solubilized prethrombin is renatured in a renaturing buffer which contains at least one reducing agent, at least one folding assistant and divalent cations. In a preferred embodiment of the invention, the solubilisate is added to the folding batch in several fractions or continuously over several days. Preferably, the solubilisate is added in a “pulse renaturing” by rapid dilution to the solubilisate. In this context, for example, 6 pulses can be performed in a time interval of 24 hours. The number of pulses is set such that after the addition of the solubilization batch the concentration of protein which has not yet been folded is not too high, since otherwise aggregates are obtained. For example, with each pulse about 0.1 g/l of protein is newly transferred into the folding batch (based on the protein concentration in the folding batch after addition of the solubilisate). A protein end concentration of 0.6 g/l can thus be achieved in the folding batch.

In a preferred embodiment of the invention, the reducing agent is an organic monosulphide. The use of glutathione (GSH) is preferred here. A mixture of GSH/GSSG can also be employed.

In a preferred embodiment of the invention, the folding assistant is chosen from arginine and glycerol. Compounds which promote the folding of proteins can generally be employed as “folding assistants”. Such compounds are known to the person skilled in the art. They can assist the folding in various ways. It is assumed that arginine destabilises incorrectly folded intermediates, so that these are at least partly unfolded again (from a thermodynamic dead-end) and therefore can be correctly folded again. On the other hand, glycerol usually stabilises proteins. Compounds which increase the absolute yield of folded prethrombin in the method according to the invention by more than 5%, in particular by more than 10 or more than 20% (based on the total amount of prethrombin employed for the folding), compared with a method without using the folding assistant, are suitable in particular as folding assistants.

The renaturing buffer comprises divalent cations. The use of calcium salts, in particular calcium chloride, is preferred. The renaturing buffer is preferably a Tris buffer.

The renaturing is preferably carried out at a pH of between 6 and 12, in particular between 7 and 11.

In a preferred embodiment, the method for producing folded prethrombin or a derivative thereof includes the following steps: Inclusion bodies which comprise non-folded prethrombin or a derivative thereof are solubilized in a solubilization buffer which comprises at least one chaotropic compound and at least one organic disulphide compound, and the solubilized prethrombin or derivative thereof is then renatured in a renaturing buffer which contains at least one reducing agent, at least one folding assistant and divalent cations, both the solubilization buffer and the renaturing buffer comprising no detergent.

In a preferred embodiment of the invention, the solubilization buffer and/or the renaturing buffer consequently contains no detergent. It has been found, according to the invention, that the use of detergents is not necessary for the solubilization and/or folding of prethrombin. This is advantageous, since certain detergents are comparatively aggressive chemical substances which pharmaceutical products should not comprise or should comprise in only small amounts and therefore must be removed in an expensive manner. The method according to the invention is therefore advantageous compared with the method of Soejima et al., 2001, in which such aggressive detergents (Triton X-100 or Brij-58) are employed for folding the protein.

The preferred absence of detergents relates not only to the solubilization and renaturing buffers referred to above, but to all the reagents and method steps employed. In other words, no detergents are used in the entire production method according to the invention, and the production method is therefore detergent-free. This relates to both aspects, both the production of folded prethrombin and the production of thrombin from this folded prethrombin.

High renaturing yields are surprisingly achieved with the method according to the invention. For example, it has been possible to achieve renaturing yields of up to 25%, based on the total prethrombin in the inclusion bodies. Preferably, a renaturing yield of at least 10% or at least 20% is achieved. The protein end concentration can be, for example, 0.6 g/l. It is preferably between 0.1 and 2 g/, in particular higher than 0.3 g/l, in order to avoid large folding volumes in the production.

By carrying out the method according to the invention with the solubilization and subsequent renaturing, an aqueous solution of folded prethrombin is obtained. The folded prethrombin can subsequently be purified further by known methods.

It has been found according to the invention that a chromatographic purification, in particular by means of hydrophobic interaction chromatography, is particularly advantageous for the further purification. The column employed can be, for example, a Phenylsepharose HP from GE Healthcare. The HIC chromatography is preferably carried out in the presence of ammonium sulphate, preferably at a concentration of 1.15 M in the high salt buffer. It has been found that under these conditions product yields of from 70 to 95% can be achieved in an elution by means of a stepwise gradient.

Preferably, the purification is not carried out by hirudin-based affinity chromatography. Preferably, no purification step with hirudin-based affinity chromatography is carried out in the method according to the invention.

Preferably, the renatured prethrombin is converted into thrombin by enzymatic proteolysis, in particular with a small venom protease, preferably ecarin. The serine protease ecarin cleaves the peptide bond between the A and B chain at the position arginine 320 and isoleucine 321 of prethrombin-2. The thrombin formed in this way becomes α-thrombin by autocatalytic splitting off of the intrinsic N terminus. In the method according to the invention, if the incubation time is sufficient thrombin is thus obtained as an intermediate product and α-thrombin as an end product. If autocatalytic cleavage is not complete, a mixture of thrombin and α-thrombin is obtained. In the following, the term “thrombin” also stands for this mixture. Proteases which have a similar functionality to ecarin can also be employed for the proteolytic cleavage. Preferably, the protease is employed in a concentration of less than 2 U/ml, preferably less than 1 U/ml or less than 0.5 U/ml.

Preferably, the thrombin and/or α-thrombin is purified by chromatography after the proteolysis. Preferably, the purification is carried out by hydrophobic interaction chromatography (HIC). The formation, caused by α-thrombin, during the process procedure of autolysis products which can form spontaneously during relatively long storage after activation of prethrombin-2 has proved to be critical. The depletion of these product-related impurities by means of ion exchange chromatography (IEX) proved to be difficult, since the pl values of α-thrombin and its autolysis products are virtually identical. When a hydrophobic interaction chromatography (HIC) was used instead, significantly more favourable depletion effects were to be observed. It is thus preferable according to the invention to carry out a purification by means of HIC on the folded prethrombin after the renaturing and on the thrombin after the proteolytic cleavage of the prethrombin. Generally, purification of cleaved prethrombin in aqueous solution or of thrombin or α-thrombin in aqueous solution by HIC offers advantages which are not limited to the present specific method of producing folded prethrombin from inclusion bodies with the features of claim 1. The invention therefore also provides generally the purification of aqueous solutions comprising folded prethrombin or thrombin or α-thrombin by means of HIC.

In a preferred embodiment of the invention, in addition to the purification by means of HC of the thrombin obtained, a further chromatographic purification step is carried out. Ion exchange chromatography (IEX) is preferably employed. By this means, the content of endotoxins, of DNA and of host cell proteins (HCP) can be reduced to a minimum.

A diafiltration with subsequent concentration of the active compound could stand at the end of the process procedure. However, other known methods can also be employed to additionally increase the purity and concentration of the thrombin.

Studies on the storage stability of α-thrombin have shown that the addition of amino acids and salt suppress autolysis and at the same time ensure the stability of a-thrombin over at least 3 months at 4° C.

In a preferred embodiment of the invention, the method according to the invention includes the following steps:
a) expression of recombinant prethrombin in prokaryotic cells and isolation of the prethrombin-containing inclusion bodies,
b) mixing of the inclusion bodies with a suitable solubilization buffer comprising at least one chaotropic substance and a disulphide compound which can form mixed disulphides with the —SH groups of the protein in the inclusion bodies,
c) removal of excess disulphide,
d) renaturing in a suitable detergent-free buffer comprising at least one reducing agent, a folding assistant and divalent cations,
e) purification of the renatured prethrombin,
f) cleavage into the active form and
g) isolation and purification of the thrombin and/or α-thrombin.

In a preferred embodiment of the invention, in step b) the mixing of the inclusion bodies in the ratio of 1:4 to 1:19 (1 g of IB paste+4 ml of solubilization buffer) with a suitable buffer having a pH in the neutral-basic range, comprising 10 mM-0.5 M GSSG, 4-8 M guanidinium hydrochloride (GuaHCl) and 0.1-10 mM EDTA takes place.

In a preferred embodiment of the invention, the mixing of the inclusion bodies in the ratio of 1:9 with a suitable buffer having a pH in the neutral-slightly basic region, comprising 0.12 M GSSG, 5 M guanidine hydrochloride (GuaHCl) and 1 mM EDTA is in step b).

In a preferred embodiment of the invention, in step c) the removal of excess reagent for the formation of mixed disulphides by a buffer change in 3-8 M, preferably 5 M guanidine hydrochloride at an acid pH (pH 3.0) is achieved.

In a preferred embodiment of the invention, the pulse renaturing in step d) is carried out with 0.01-1.0 g/l of protein (based on the protein concentration in the folding batch per pulse) in a suitable detergent-free buffer having a neutral-slightly basic pH, comprising 0.1-3 mM GSH, 0.1-2 M arginine, 0.001-1M CaCl₂, 0.1-50 mM EDTA and 1-40% glycerol.

In a preferred embodiment of the invention, step d) is carried out by rapid dilution of 0.1 g/l of solubilisate in a suitable detergent-free buffer having a neutral-slightly basic pH, comprising 0.75 mM GSH, 1M arginine, 50 mM CaCl₂, 1 mM EDTA and 20% glycerol, the protein concentration in the folding batch increasing by 0.1 g/l per pulse and 6 pulses being performed with an interval of in each case 24 h.

The present invention describes a novel prokaryotic method for producing thrombin on an industrial scale, in which the yield of prethrombin is increased several-fold and the outlay and the costs are therefore reduced significantly compared with known methods. The increased yield is achieved in particular by novel solubilization and renaturing methods.

### FIGURES

FIG. 1: shows the purity of prethrombin-2 after renaturing in SDS-PAGE gel. The renaturing of prethrombin-2 was carried out by the pulse renaturing method up to a protein end concentration of 0.6 g/l. Renaturing yields of up to 25% were achieved. The renaturing batch in Coomassie-stained SDS-PAGE gel is shown.

FIG. 2: shows the purification of α-thrombin via hydrophobic interaction chromatography (Toyopearl Butyl-650S). The filtered ecarin cleavage batch (4.5 ml+4.5 ml of high salt buffer: 2 M ammonium sulphate, 50 mM phosphate buffer pH 6.0) was purified over a 4 ml HIC column (Tricorn 5/200). The equilibration was carried out with 50% high salt buffer and the elution by means of linear gradients over 20 CV to 50 mM phosphate buffer pH 6.0.

FIG. 3: shows the amino acid sequence, the structure and important properties of prethrombin-2.

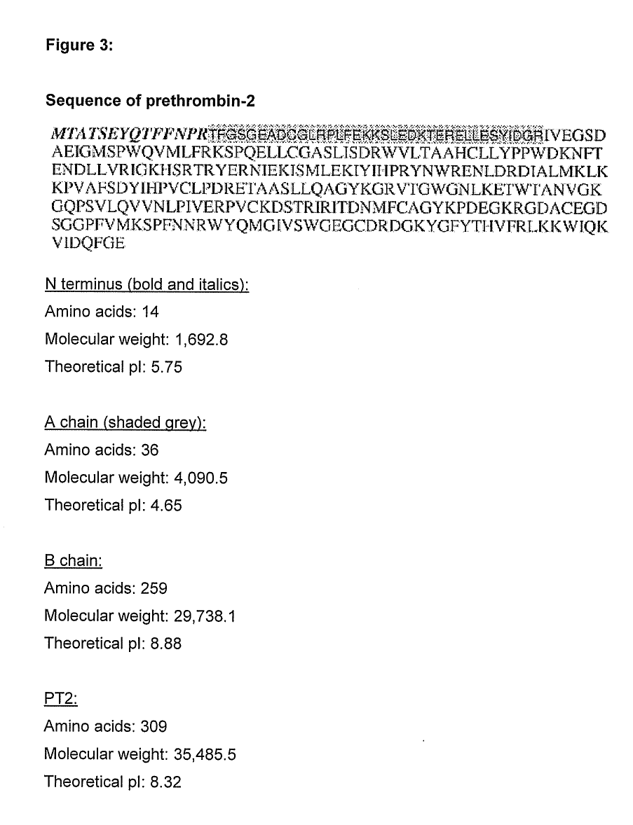

FIG. 4: shows the amino acid sequence, the structure and important properties of alpha-thrombin.

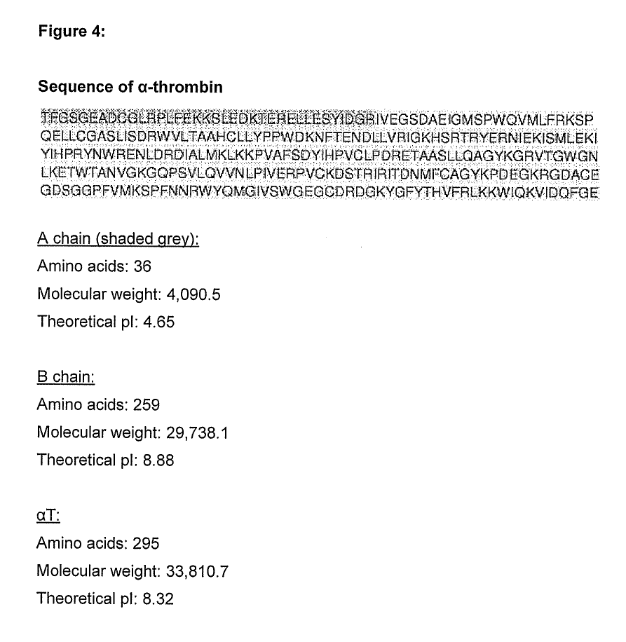

### EMBODIMENT EXAMPLES

### Example 1

### Expression of rh-prethrombin-2

The bacterial host E. coli JM108 used for expression of rh-prethrombin-2 (DSMZ 5585; F⁻ thi Δ (lac-proAB) end Al gyrA96 re/Al phx hsdRI7 supE44 recA) is proline-auxotrophic, which was neutralized by the use of the plasmid with the designation pSCIL048. The plasmid pSCIL048 is based on the plasmid pSCIL008 (see WO05061716). The strain cannot synthesise thiamine (Vieira & Messing, 1982 Gene. October; 19(3):259-68). Prethrombin-2 is expressed under the control of the tac promoter located on pSCIL048. The vector pSCIL048 used here is a high copy plasmid with a kanamycin resistance. The expression is carried out in defined mineral salt medium and is induced by the addition of IPTG. The prethrombin-2 is deposited in the cytosol in the form of inclusion bodies (IBs).

The biomass production was carried out at 37° C. The aim of this fermentation was to obtain product and biomass for subsequent process steps. To monitor the overexpression of the target protein during the fermentation process, samples were analysed by means of SDS-PAGE before and after induction. A biomass-specific increase in the protein concentration was to be observed up to 3 h after induction.

### Example 2

### Cell Breakdown and Preparation of Inclusion Bodies (IB)

The expression of the target protein prethrombin-2 took place in the form of IBs. The cell breakdown and the IB preparation were carried out in accordance with standard protocols and can be conducted on the laboratory scale up to a working up of approx. 200 g of biomass.

### Example 3

### Solubilization and Renaturing

In the optimised renaturing protocol according to the invention, the re-folding was carried out on the basis of mixed disulphides.

For preparation of the mixed disulphides, the IBs were homogenised in a ratio of 1 g of IB paste+9 ml of solubilization buffer with 5 M GuaHCl; 0.1M Tris-HCl; 1 mM EDTA; 0.1M GSSG pH 8.5 and solubilized at RT for 3 h. After a centrifugation step at 50,000×g over 30 min, a rebuffering step was carried out in 5 M GuaHCl; 1 mM HCl pH 3.0 to separate off free GSSG/GSH mixture.

After a centrifugation step at 50,000×g over 30 min (optional), a rebuffering step was carried out in 5 M GuaHCl (3-8 M), 1 mM HCl pH 3.0 (acid pH is important if the solubilisate is not added directly thereafter to the folding batch) to separate off free GSSG/GSH mixture.

The pulse renaturing was carried out by rapid dilution of the solubilisate in the folding buffer 1M arginine, 50 mM Tris, 50 mM CaCl₂, 1 mM EDTA, 20% glycerol, 0.75 mM GSH pH 8.5, up to 6 pulses in a time interval of 24 h preferably being performed. 0.1 g/l of protein per pulse was newly transferred into the folding batch (based on the protein concentration in the folding batch after addition of the solubilisate). A protein end concentration of 0.6 g/l was achieved in the folding tank.

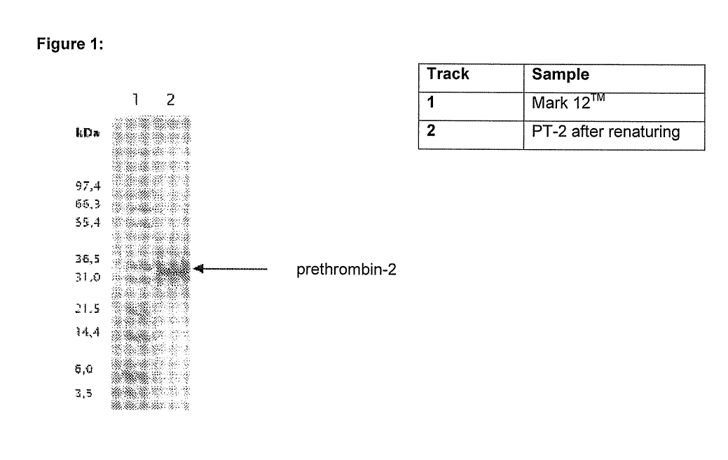

FIG. 1 shows the purity of the product after the renaturing. Renaturing yields of 25%, based on the amount of solubilisate introduced into the folding batch, were achieved by this method.

### Example 4

### Purification of Prethrombin-2

Since relatively high losses in yield occurred during exchange by means of diafiltration, ammonium sulphate was added instead (preferably an end concentration of 1.15 M). The precipitation precipitate was separated off by means of centrifugation and the supernatant was applied to an HIC column (preferably Phenyl Sepharose HP, GE Healthcare), in order then to elute prethrombin in the desired buffer.

### Example 5

### Activation of Prethrombin

The activation of prethrombin-2 to give α-thrombin was carried out with the serine protease ecarin, which cleaves specifically the peptide bond between the A and B chain (Arg₃₂₀-Ile₃₂₁) of prethrombin-2. From the thrombin formed in this way, α-thrombin is formed by autocatalytic splitting off of the intrinsic N terminus. The cleavage conditions were chosen such that a maximum cleavage yield is achieved with the lowest possible ecarin requirement. In this context, 2,000 μg of prethrombin-2 (0.5-0.8 mg/ml) were cleaved with 1 U of ecarin in a vibrating incubator in 24 h at 37° C. and 600 rpm. The reaction was stopped by addition of EDTA to an end concentration of 25 mM. After the cleavage, precipitated protein was observed, and the solution was therefore filtered (0.2 μm) before the purification by chromatography. The cleavage yield was between 70% and 90%, depending on the amount of precipitate.

### Example 6

### Purification of α-thrombin

The filtered ecarin cleavage batch was used as the starting material for the purification by chromatography. The aim of the purification was, in addition to separating off of host cell proteins, above all the depletion of non-cleaved prethrombin-2 and of the autocatalytic cleavage products of α-thrombin. At the time of the ecarin cleavage, prethrombin-2 is present in a purity of more than 95%. For better investigation of the depletion of the cleavage products, these were concentrated by incubation of the cleavage batches at 37° C. for a further 24 h.

Separating off the thrombin derivates by means of ion exchange chromatography proved to be difficult, since they have virtually identical pl values (pl ˜9) The pl values of α-thrombin and its autolysis products were determined experimentally by means of 2D gel electrophoresis. In addition to cation exchange materials, hydrophobic interaction chromatography materials were therefore tested.

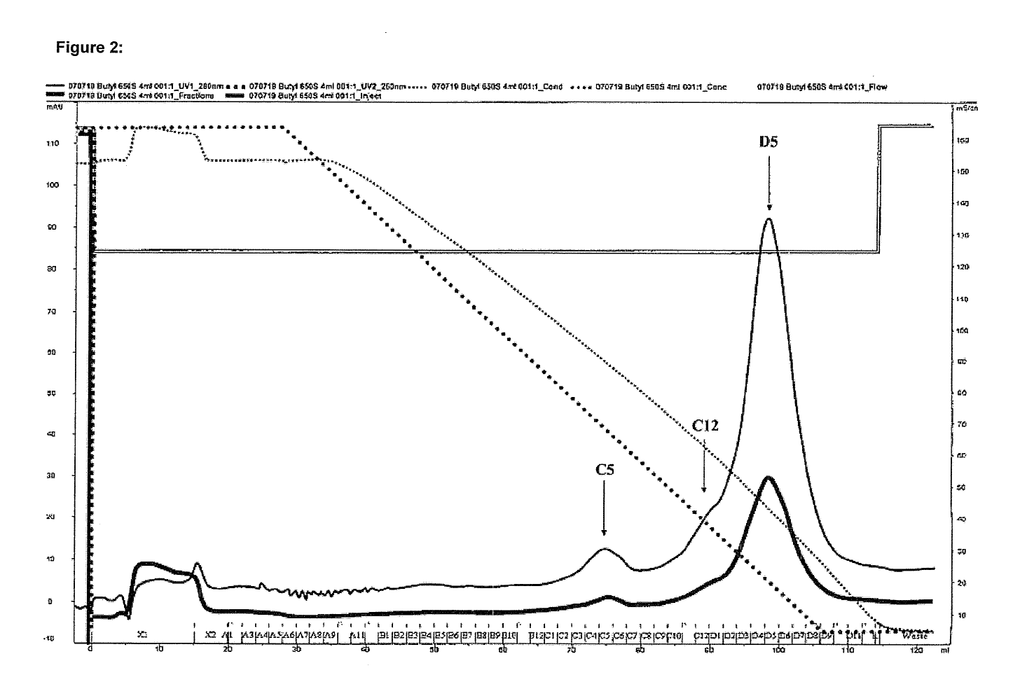

FIG. 2 shows the chromatogram of a purification over a 4 ml HIC column (Toyopearl Butyl-650S). For this, before application to the column 4.5 ml of the filtered ecarin cleavage batch were diluted 1:1 with high salt buffer (2 M ammonium sulphate, 50 mM phosphate buffer pH 6.0). The elution was carried out by means of a linear gradient (starting with 50% high salt buffer) to 50 mM phosphate buffer pH 6.0 over 20 CV. In order to investigate the result of the purification, directly after the chromatography PMSF was added to the main fractions up to an end concentration of 1 mM in order to inhibit further autolysis. In total, the yield of α-thrombin in the main peak is 65% (determined with UV₂₈₀).

### Result:

In the method according to the invention for producing rh-thrombin in E. coli, the yields described in the literature are exceeded significantly. Thus, the product yields described in the literature for native prethrombin-2 in eukaryotic expression systems are between 25 and 200 mg/l of fermentation medium (Russo et al. 1997, Yonemura et al. 2004).

In contrast, the yields of native prethrombin-2 achieved according to the invention are 400 mg/l of fermentation medium. Yields of α-thrombin of 200 mg/l of fermentation medium with an rp-HPLC purity of at least 95% are achieved. Furthermore, the prokaryotic expression system employed is simpler to establish than known eukaryotic methods.

In the prokaryotic method described by Soejima et al., 2001, folding yields of native prethrombin-2 after renaturing of 4-7% are achieved. In the method filed according to the invention, on the other hand, 25% folding yields were obtained. The volume of the folding batch used was moreover reduced (increase in the protein concentration in the folding)

1. A method for producing folded prethrombin or a derivative thereof, wherein inclusion bodies which comprise non-folded prethrombin or a derivative thereof are solubilized in a solubilization buffer which comprises at least one chaotropic compound and at least one organic disulphide compound, and the solubilized prethrombin or derivative thereof is then renatured in a renaturing buffer which contains at least one reducing agent, at least one folding assistant and divalent cations, both the solubilization buffer and the renaturing buffer comprising no detergent.

2. The method according to claim 1, wherein the disulphide compound is glutathione disulphide.

3. The method according to claim 1 or 2, wherein the chaotropic compound is chosen from guanidinium salts, iodides, barium salts, thiocyanates, urea and perchlorates.

4. The method according to claim 1, wherein the inclusion bodies have been obtained by recombinant expression of prethrombin or a derivative thereof in prokaryotic cells.

5. The method according to claim 1, wherein the reducing agent is an organic monosulphide.

6. The method according to claim 1, wherein the folding assistant is chosen from arginine and glycerol.

7. The method according to claim 1, wherein the folding is carried out in a pulse renaturing method.

8. The method according to claim 1, wherein the renatured prethrombin or derivative is purified by chromatography.

9. The method according to claim 8, wherein the purification is carried out by hydrophobic interaction chromatography (HIC).

10. A method for producing thrombin and/or α-thrombin or a derivative thereof, wherein the renatured prethrombin produced according to claim 1 is converted into thrombin and/or α-thrombin or the derivative thereof by enzymatic proteolysis, in particular with ecarin.

11. The method according to claim 10, wherein the thrombin and/or α-thrombin or derivative thereof is purified by chromatography after the proteolysis.

12. The method according to claim 11, wherein the purification is carried out by hydrophobic interaction chromatography (HIC).

13. A solution comprising folded prethrombin, folded thrombin and/or folded α-thrombin, or a derivative thereof, obtainable by a method according to claim 1.

14. The method according to claim 3, wherein the chaotropic compound is chosen from guanidinium hydrochloride and guanidinium thiocyanate.

15. The method of claim 10, wherein the conversion by enzymatic proteolysis is done with ecarin.


SEQUENCE LISTING (2 sequences)

SEQ ID NO: 1 - protein, 309 aa, Homo sapiens
MTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 2 - protein, 295 aa, Homo sapiens
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE